(19) United States
(12) Patent Application Publication
(10) Pub. No.: US 2009/0275552 A1
(43) Pub. Date: Nov. 5, 2009

(54) Therapy for Treating Resistant Bacterial Infections

(76) Inventors: Mahesh Vithalbhai Patel, Maharashtra (IN); Shrikant Vinayak Gupte, Maharashtra (IN); Sachin Subhash Bhagwat, Maharashtra (IN); Mohammad Alam Jafri, Maharashtra (IN); Girish Kumar Jain, Delhi (IN); Mandar Madhukar Kodgule, Maharashtra (IN)

Correspondence Address: BIO INTELLECTUAL PROPERTY SERVICES (BIO IPS) LLC, 8509 KERNON CT., LORTON, VA, 22079, US

(21) Appl. No.: 12/226,525

(22) PCT Filed: Apr. 27, 2007
(86) PCT No.: PCT/IB2007/001104

§ 371 Date: Jun. 1, 2009

(30) Foreign Application Priority Data
Apr. 28, 2006 (IN) 672/MUM/2006
Apr. 28, 2006 (IN) 673/MUM/2006

(51) Int. Cl. A61K 31/397

(52) U.S. Cl. 514/210.05

(57) ABSTRACT

The invention relates to an improved therapy for treating resistant bacterial infections caused by extended-spectrum β-lactamase (ESBLs)-producing strains in a warm-blooded animal, adjuvant step down therapy, and pharmaceutical compositions for such therapies. The invention also relates to a method for inhibiting bacterial resistance in ESBLs-producing strains so as to have better control over the therapy; achieve reduced hospital stay and adjuvant step down therapy so as to avoid recrudescence. In particular, the therapy includes antibacterial combination of cefepime with sulbactam via parenteral route, followed by oral third generation cephalosporin with a suitable β lactamase inhibitor.

### FIELD OF THE INVENTION

The invention relates to an improved therapy for treating resistant bacterial infections caused by extended-spectrum β-lactamase (ESBLs)-producing strains in a warm blooded animal, adjuvant step down therapy, and pharmaceutical compositions for such therapies. The invention also relates to a method for inhibiting bacterial resistance in ESBLs-producing strains so as to have better control over the therapy; achieve reduced hospital stay and adjuvant step down therapy so as to avoid recrudescence. In particular, the therapy includes antibacterial combination of cefepime with sulbactam via parenteral route, followed by oral third generation cephalosporin with a suitable β lactamase inhibitor.

### BACKGROUND OF THE INVENTION

Cefepime is a semi-synthetic, broad spectrum, fourth generation cephalosporin antibiotic. Cefepime is commercially available as hydrochloride salt (Formula I) under the trade name of Maxipime®. Chemically, it is 1-[[(6R,7R)-7-[2-(2-amino-4-thiazolyl)-glyoxy]amido]-2-carboxy-8-oxo-5-thia-1-azabicyclo[4.2.0]oct-2-en-3-yl]methyl]-1-methylpyrrolidinium chloride, 7² (Z)-(O-methyloxime), monohydrochloride monohydrate.

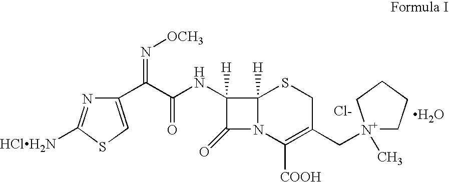

Cefepime exerts antibacterial functions on G+ve bacteria, such as Staphylococcus aureus, Staphylococcus epidermidis, Streptococcus pyogenes, pathogenesis staphylococcal bacteria, Streptococcus pneumoniae and other hemolytic streptococcus etc. It also has good antibacterial functions on G-ve bacteria, such as Pseudomonas aeruginosa, Escherichia coli, Klebsiella, Enteric bacilli, Bacillus proteus, Hemophilus, neisseria, Salmonella, serratia, Shigaella, and Yersinia, etc., but it is ineffective against P. Maltophilia. In addition, it has good antibacterial functions on anaerobic bacteria, such as bacteroid and Cl. perfringens, etc. but it is ineffective to Bacteroides fragilis and Clostridium difficile.

Cefepime follows linear pharmacokinetics over the range of 250 mg-2 g (IV) and 500 mg-2 g (IM). Its average steady state Vd is 18.0 (±2.0) L and serum protein binding is approximately 20%. It is principally eliminated via renal excretion, average (±SD) half-life of cefepime is 2.0 (±0.3) hours and total body clearance is 120.0 (±8.0) mL/min. It is metabolized to N-methylpyrrolidine (NMP), which is rapidly converted to the N-oxide (NMP-N-oxide). Cefepime hydrochloride is indicated in the treatment of infections like pneumonia (moderate to severe), uncomplicated and complicated urinary tract infections (including pyelonephritis), uncomplicated skin and skin structure and complicated intra-abdominal infections (used in combination with metronidazole).

Sulbactam, a derivative of the basic penicillin nucleus, is an irreversible beta-lactamase inhibitor. Sulbactam is commercially available as sodium salt (Formula II) in combination with ampicillin (β lactam antibiotic) under the trade name of Unasyn®. Chemically, it is (2S,5R)-3,3-dimethyl-7-oxo-4-thia-1-azabicyclo[3.2.0]heptane-2-carboxylate 4,4-dioxide. The mean serum half-life of sulbactam is approximately 1 hour and approximately 75 to 85% of sulbactam is excreted unchanged in the urine. It is given in combination with beta-lactam antibiotics to overcome beta-lactamase enzyme that destroys the antibiotics.

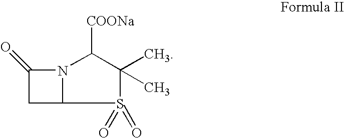

### Third-Generation Oral Cephalosporins

These drugs have excellent activity against Enterobacteriaceae. Orally active third generation cephalosporins include cefdinir, cefditoren, cefixime, cefpodoxime, cefprozil and ceftibuten.

Cefdinir having Formula III is an extended-spectrum, semisynthetic cephalosporin, for oral administration. Cefdinir is commercially available under the trade name of Omnicef®. Chemically, it is [6R-[6_{—},7_(Z)]]-7-[[(2-amino-4-thiazolyl) (hydroxyimino)acetyl]amino]-3-ethenyl-8-oxo-5-thia-1-azabicyclo[4.2.0]oct-2-ene-2-carboxylic acid.

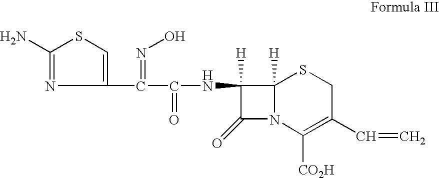

Cefditoren pivoxil is a semi-synthetic cephalosporin for oral administration. It is a prodrug, which is hydrolyzed by esterases during absorption and the drug is distributed in the circulating blood as active cefditoren. Cefditoren pivoxil is commercially available under the trade name of Spectracef®. Chemically, cefditoren pivoxil (Formula IV) is (−)-(6R,7R)-2,2-dimethylpropionyloxymethyl 7-[(Z)-2-(2-aminothiazol-4-yl)-2-methoxyiminoacetamido]-3-[(Z)-2-(4-methylthiazol-5-yl)ethenyl]-8-oxo-5-thia-1-azabicyclo[4.2.0]oct-2-ene-2-carboxylate.

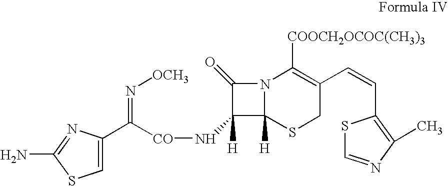

Cefixime of Formula V is a semisynthetic, cephalosporin antibiotic for oral administration. Cefixime is commercially available under the trade name of Suprax®. Chemically, it is (6R,7R)-7-[2-(2-Amino-4-thiazolyl)glyoxy]amido]-8-oxo-3-vinyl-5-thia-1-azabicyclo[4.2.0]oct-2-ene-2-carboxylic acid, 7²-(Z)-[O-(carboxymethyl)-oxime]trihydrate.

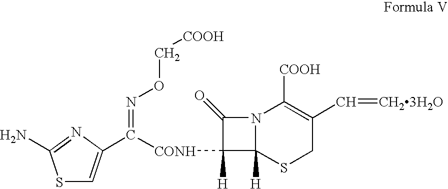

Cefpodoxime proxetil of Formula VI is an orally administered extended spectrum, semi-synthetic antibiotic of the cephalosporin class. Cefpodoxime proxetil is commercially available under the trade name of Vantin®. Chemically, it is (RS)-1(isopropoxycarbonyloxy)ethyl(+)-(6R,7R)-7-[2-(2-amino-4-thiazolyl)-2-{(Z) methoxyimino}acetamido]-3-methoxymethyl-8-oxo-5-thia-1-azabicyclo[4.2.0]oct-2-ene-2-carboxylate.

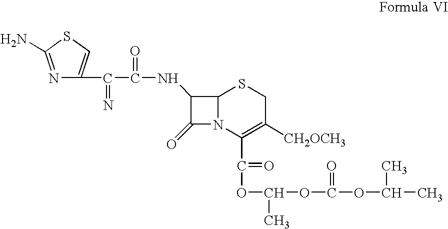

Ceftibuten dihydrate of Formula VII is a semisynthetic cephalosporin antibiotic for oral administration. Ceftibuten dihydrate is commercially available under the trade name of Cedax®. Chemically, it is (+)-(6R,7R)-7-[(Z)-2-(2-Amino-4-thiazolyl)-4-carboxycrotonamido]-8-oxo-5-thia-1-azabicyclo[4.2.0]oct-2-ene-2-carboxylic acid, dihydrate.

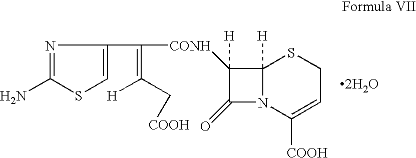

Cefprozil of Formula VIII is a cis and trans isomeric mixture (≧90% cis). Cefprozil is commercially available under the trade name of Cefzil®. Chemically, it is (6R,7R)-7-((R)-2-amino-2-(p-hydroxy-phenyl)acetamido)-8-oxo-3-propenyl-5-thia-1-azabicyclo(4.2.0)oct-2-ene-2-carboxylic acid monohydrate.

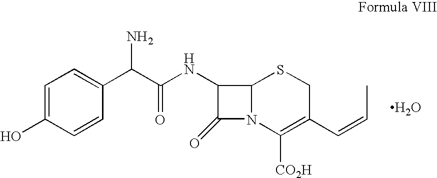

Tazobactam sodium of Formula IX, a derivative of the penicillin nucleus, is a penicillanic acid sulfone. Tazobactam is commercially available as sodium salt in combination with Piperacillin under the trade name of Zosyn®. Chemically, it is sodium (2 S,3 S,5R)-3-methyl-7-oxo-3-(1H-1,2,3-triazol-1-ylmethyl)-4-thia-1-azabicyclo[3.2.0]heptane-2-carboxylate-4,4-dioxide.

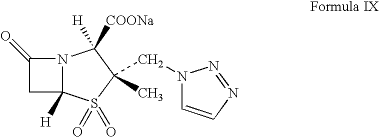

CN Publication No 1565456A discloses an antibacterial combination composed of cefepime and beta-lactamase depressant in the weight ratio from 1:2˜1:0.1. The combination of beta-lactamase depressant and cefepime described in this invention has obvious combined antibacterial functions. The various dose combinations of cefepime with sulbactam that have been exemplified in this publication are given in table 1.

TABLE I
The various dose combinations of cefepime with sulbactam.

| Practical | Content Per Vial | |
|---|---|---|
| Example | Cefepime/Cefepime | |
| numbers as given | hydrochloride/ | |
| in publication No | Cefepime hydrochloride | Sulbactam Sodium |
| CN 1565456A | hydrate (gm) | (gm) |
| 5 | 1 | 0.1 |
| 6 | 1 | 1 |
| 7 | 0.5 | 1 |
| 8 | 1 | 0.75 |
| 15 | 1 | 0.5 |
| 16 | 1 | 2 |
| 26 | 1 | 2 |
| 27 | 1 | 2 |
| 28 | 1 | 0.2 |
| 29 | 1.11 | 0.33 |

CN Publication No 1565455A discloses a kind of bacteriophage combination for curing the infection caused due to the bacteria responsible for extended spectrum beta-lactamase. It is composed of cefepime and tazobactam with their weight ratio from 20:1˜1:2.

### SUMMARY OF THE INVENTION

In one general aspect there is provided a method of inhibiting bacterial infections caused by resistant Extended-spectrum β-lactamase producing strains (ESBLs) in a warm-blooded animal. The method includes administering a combination of 2 gm of cefepime and 0.1-4 gm of sulbactam to the warm-blooded animal.

In another general aspect there is provided a pharmaceutical composition that includes 2 gm of cefepime in combination with 0.1-4 g of sulbactam and one or more pharmaceutically acceptable excipients.

Embodiments of the composition may include one or more of the following features. For example, the composition may be administered parenterally.

In another general aspect there is provided a method for treating a resistant bacterial infections caused by Extended-spectrum β-lactamase producing strains (ESBLs) in a warm blooded animal, the method comprising providing a dosage form to the warm blooded animal comprising cefepime in combination with sulbactam via parenteral route, followed by providing a dosage form that includes an oral third generation cephalosporin with a suitable β lactamase inhibitor.

Embodiments of the method may include one or more of the following features. For example, the oral third generation cephalosporin and β lactamase inhibitor may be present in a ratio of 1:1 to 1:4.

The oral third generation cephalosporin may include one or more of cefdinir, cefditoren, cefixime, cefpodoxime, cefprozil ceftibuten, and the like along with a suitable β lactamase inhibitor selected from sulbactam, tazobactam, and the like. The oral dosage form may be in the form of tablets, powder, capsules or granules to be reconstituted before administration

The details of one or more embodiments of the inventions are set forth in the description below. Other features, objects and advantages of the inventions will be apparent from the description and claims.

### DETAILED DESCRIPTION OF THE INVENTION

Antibacterials are the agents used to inhibit or kill the pathogenic bacteria and many a time these pathogenic bacteria develop resistance against antibacterials by one or more mechanisms. As a result, efficacy of antibacterials gets reduced and bacteria become ineffective towards them. To overcome this problem, the antibacterial levels are required to be kept same and the resistance level need to be reduced by some means so that resistant organisms become sensitive towards these antibacterials.

It is well known in the prior art that the combination of cefepime with sulbactam has solved the increasing problem of clinical pathogenic bacteria having drug resistance to cefepime. The inventors have surprisingly found that when 2 g of cefepime i.e. maximum permissible dose is combined with 0.1-4 g of sulbactam, it shows enhanced efficacy over 1 g cefepime and 0.1-2 g sulbactam combination. Also, it was surprisingly found that when 2 g of cefepime is combined with 0.1-4 g sulbactam, it is effective in inhibiting the resistance in ESBLs producing strains. The term “ESBLs” refers to Extended Spectrum Beta-Lactamase producing strains.

In warm-blooded animal with long-standing severe infections related to resistant ESBL strain, antibiotic therapy is continued for longer period so as to avoid recrudescence. There are several limitations of parenteral therapy. For example, administration by a skilled or a trained person, need for a sterile dosage form, and pain and cell necrosis associated with administration. All of these factors contribute to increased hospital stay, which in turn increases the expenses. The present inventors while working on the above problem have come up with an adjuvant step down oral therapy.

A first aspect of the invention provides a method of inhibiting bacterial infections caused by resistant ESBL producing strains in a warm blooded animal. The method includes administering a combination of 2 gm of cefepime and 0.1-4 gm of sulbactam to a warm blooded animal.

Embodiments of the invention include one or more of the following studies. For establishing the efficacy of the combination of the present invention, several in-vitro microbiological studies have been preformed. In one such study, cefepime alone was tested for inhibition of growth of the ESBL producing strains of known pathogens. The study is described in detail in Example 1. The study indicates that when higher concentration of cefepime is achieved, the minimum inhibitory concentrations (MICs) required for the inhibition of growth of resistant ESBL-producing strain have been surprisingly found to drop from 16 μg/ml to less than 0.03 μg/ml (refer Table 1). This higher concentration of cefepime is not achieved by 1 gm of cefepime for more than 2 hours and therefore the resistance to ESBL-producing strain develops, thus rendering the 1 gm dose in combination with cefepime to be ineffective in treating such infections.

In another study, the inventors have found that the higher concentration of cefepime in the blood, which is achievable only by 2 gm dose for maximum of 6 hours in combination with fixed dose of sulbactam, is effectively capable of inhibiting resistant bacterial zone 25% more than 500 mg dose and 15% more than 1 gm dose in combination with fixed dose of sulbactam.

In yet another study, the inventors have found that the higher concentration of cefepime in the blood, which is achievable only by 2 gm dose for maximum of 6 hours in combination with fixed dose of sulbactam, inhibits the growth almost 500 times more efficiently than compared with 500 mg of cefepime and 92 times more effective over 1 gm of cefepime in combination with fixed dose of sulbactam.

A second aspect of the invention provides a pharmaceutical composition comprising 2 gm of cefepime in combination with 0.1-4 g of sulbactam and one or more pharmaceutically acceptable excipients.

Embodiments of the invention may include one or more of the following features. The two components of dosage form; viz., cefepime and sulbactam can be lyophilized separately and filled in one vial or individually can be lyophilized and kept in separate vials. Before administration to the warm-blooded animal in need thereof, the contents of the individual vial are reconstituted using suitable pharmaceutically acceptable diluents and administered. Cefepime may be present in the form of cefepime internal salt, cefepime hydrochloride or cefepime hydrochloride hydrate, or the addition agents like L-arginine. Sulbactam may be present as sodium or potassium salt. The in vitro studies show that the dosage form containing 2 gm of cefepime (maximum permissible dose) and 0.1-4 g of sulbactam show enhanced bactericidal activity over 1 g cefepime with 0.1-2 g sulbactam combination. The bactericidal activity is found to increase proportionally with proportional increase in cefepime concentration. The pharmaceutical antibacterial composition may also contain one or more pharmaceutically acceptable excipients. The pharmaceutically acceptable excipients may include one or more of antioxidants, buffers, preservatives, tonicity agents, chelating agents, and the like.

Suitable-antioxidants include one or more of butylated hydroxytoluene (BHT), ascorbic acid, sodium bisulphite, sodium metabisulphite, and the like. Suitable buffers include one or more of citrates, acetates, borax, phosphates, and the like.

Suitable preservatives include one or more of benzyl alcohol, methyl paraben, propyl paraben, benzyl paraben, and the like. Suitable tonicity agents include one or more of dextrose, sodium chloride, mannitol, and the like. Suitable chelating agents include one or more of sodium ethylene-diamine-tetra-acetic acid (EDTA), citric acid, and the like.

A third aspect of the invention provides a method of treating a resistant bacterial infections caused by extended-spectrum β-lactamase producing strains (ESBLs) in a warm blooded animal, the method comprising providing a dosage form to the warm blooded animal comprising cefepime in combination with sulbactam via parenteral route, followed by providing a dosage form that includes an oral third generation cephalosporin with a suitable β lactamase inhibitor.

The oral third generation cephalosporin and β lactamase inhibitors may be present in a weight ratio of 1:1 to 1:4.

Suitable oral third generation cephalosporin may include one or more of cefdinir, cefditoren, cefixime, cefpodoxime, cefprozil ceftibuten, and the like. Suitable β lactamase inhibitor may include one or more of sulbactam, tazobactam, and the like.

The oral dosage form may include tablets, powder, capsule or granules to be reconstituted before administration.

For the purpose of this disclosure, a warm-blooded-animal is a member of the animal kingdom possessed of a homeostatic mechanism and includes mammals and birds.

While the present invention has been described in terms of its specific embodiments, certain modifications and equivalents will be apparent to those skilled in the art and are intended to be included within the scope of the present invention.

### Example 1

TABLE 1
Effect of sulbactam on Minimum Inhibitory Concentrations (MICs) of
cefepime against ESBL producing strains.

| | | | Cefepime + Sulbactam | | |
|---|---|---|---|---|---|
| | | Cefepime | MICs (μg/ml) | | |
| Strains | Source | MICs (mcg/ml) | 2 | 4 | 8 |
| E. coli 1 | Metropolis | 16 | 4 | 0.25 | <0.03 |
| E. coli 17 | Metropolis | 16 | 4 | 1 | 0.06 |
| E. coli 215 | JRH | 16 | 2 | 1 | 0.06 |
| E. coli 537 | JRH | 16 | 2 | 0.25 | 0.06 |
| E. coli 548 | JRH | 16 | 2 | 0.25 | 0.06 |
| Klebsiella 555 | JRH | 16 | 4 | 1 | 0.06 |
| Klebsiella 524 | JRH | 16 | 4 | 1 | 0.06 |
| Klebsiella 624 | JRH | 16 | 4 | 0.5 | <0.03 |
| Klebsiella 583 | JRH | 16 | 8 | 4 | 0.06 |

Procedure: MICs were determined as per NCCLS recommendations using Mueller Hinton Agar (MHA, Difco, USA). 20 ml of warm molten MHA containing serial two fold dilutions of cefepime with and without sulbactam was poured in to 90 mm diameter petridishes. Sulbactam at 2, 4 and 8 mcg/ml concentrations was added to various 2-fold concentrations of cefepime. The plates were allowed to solidify at room temperature. Bacterial strains were grown in TSB (Tryptic soya broth, Difco, USA) overnight and diluted to approximately to 10⁷ CFU/ml. These culture dilutions were delivered on agar surface so as to give 10⁴ CFU per spot using a device called multipoint inoculator (Applied Quality Services, UK). Incubation of plates was done at 37° C., and growth was scored after 24 h. Quality control strain of E. coli ATCC 25922 was used as internal standard. Control growth medium was devoid of any anti-bacterial agent.

### Example 2

TABLE 2
Effect of increasing concentrations of cefepime with constant
sulbactam concentration on enhancement of antibacterial
action against cefepime resistant E. coli 71 MP.

| | Zone of | | Zone of |
|---|---|---|---|
| Cefepime | inhibition | Cefepime + sulbactam | inhibition |
| mcg/disc | (mm) | (mcg/disc) | (mm) |
| 0.5 | Nil | 0.5 + 5 | 10 |
| 1 | Nil | 1 + 5 | 11 |
| 2 | Nil | 2 + 5 | 12 |
| 4 | Nil | 4 + 5 | 13 |
| 8 | Nil | 8 + 5 | 14 |
| 16 | Nil | 16 + 5 | 15 |

Procedure: The experiment was performed by quantitative agar drug diffusion assay. 50 ml of fresh sterile molten Mueller Hinton Agar (MHA, Difco) was poured in sterile petri plate positioned on a leveled surface. Plates containing media were allowed to cool at 4° C. Bacterial inoculum was spread using sterile cotton swab, which was pre-dipped in a CFU (Colony forming units) adjusted bacterial suspension. The discs containing cefepime alone and cefepime with sulbactam were firmly placed on to culture seeded agar surface under sterile conditions. The plates were incubated at 37° C. for 24 h. The diameter of zones of inhibition was measured and recorded.

### Example 3

TABLE 3
Mutant Prevention Concentration (MPC) of cefepime with and
without sulbactam using cefepime resistant E. coli 71 MP

| | | No of colonies | No of colonies |
|---|---|---|---|
| | | per agar plate | per agar plate |
| Cefepime | Cefepime | Cefepime + | Cefepime + |
| Conc. | alone | 4 mcg/ml of | 8 mcg/ml of |
| (mcg/ml) | Type of growth | sulbactam | sulbactam |
| 4 | Mat growth | 564 | 92 |
| 8 | Mat growth | 130 | Nil |
| 16 | Mat growth | Nil | Nil |

Procedure: Overnight grown cultures of cefepime resistant Gram-negative bacteria were brought to a log phase and concentrated in normal saline to a cell density of 5×10⁹ CFU/ml by centrifugation. 150 μl of this suspension was spread in triplicate on to a large petri plates containing Mueller Hinton agar (MHA, Difco, USA). Plates were prepared with 4, 8, and 16 mcg/ml cefepime alone and each of these concentrations in combination with 4 and 8 mcg/ml of sulbactam. Plates were incubated at 37° C. for 48 hours and resistant colonies were enumerated.

### Example 4-12

TABLE 4
cefepime and sulbactam combinations composition.

| | Cefepime/Cefepime | | | Sterile |
|---|---|---|---|---|
| | Hydrochloride/ | | | water for |
| | Cefepime Hydrochloride | | | injection/ |
| | Hydrate | | | normal |
| | (Calculated on the basis | Sulbactam | | saline/5% |
| Example | of free | sodium | L-arginine | dextrose |
| No. | Cefepime base in gm) | (gm) | (gm) | (mL) |
| 4 | 20 | 2 | 14.5 | q.s |
| 5 | 20 | 5 | 14.5 | q.s |
| 6 | 20 | 10 | 14.5 | q.s |
| 7 | 20 | 15 | 14.5 | q.s |
| 8 | 20 | 20 | 14.5 | q.s |
| 9 | 20 | 25 | 14.5 | q.s |
| 10 | 20 | 30 | 14.5 | q.s |
| 11 | 20 | 35 | 14.5 | q.s |
| 12 | 20 | 40 | 14.5 | q.s |

Procedure: As mentioned in the examples (i.e. Example Nos. 4-12), suitable quantity of Cefepime/Cefepime Hydrochloride/Cefepime Hydrochloride Hydrate, sulbactam sodium and L-arginine were mixed under aseptic and clean conditions and proceeded according to the preparation process sequence of dry powder injection preparation to prepare 10 units of powder injection to be reconstituted with suitable solvents like sterile water for injection, normal saline, 5% dextrose before administration.

### Example 13

4 units of cefepime hydrochloride for injection (dosage: 0.5 g/bottle) and sulbactam sodium for injection (dosage: 2.0 g/bottle) were dissolved in the same 250 ml glucose transfusion under aseptic and clean conditions for the intravenous guttae of patients infected with sensitive bacteria.

### Example 14-19

TABLE 5
cefepime and sulbactam combinations

| | Cefepime/Cefepime | | | |
|---|---|---|---|---|
| | Hydrochloride/ | | | |
| | Cefepime | | | |
| | Hydrochloride | | | Sterile water |
| | Hydrate (Calculated | | | for injection/ |
| | on the basis | Sulbactam | | normal saline/ |
| Example | of free Cefepime | sodium | L-arginine | 5% dextrose |
| No. | base in gm) | (gm) | (gm) | (mL) |
| 14 | 5 | 0.5 | 3.625 | q.s |
| 15 | 5 | 2.5 | 3.625 | q.s |
| 16 | 5 | 5 | 3.625 | q.s |
| 17 | 10 | 10 | 7.25 | q.s |
| 18 | 10 | 15 | 7.25 | q.s |
| 19 | 10 | 20 | 7.25 | q.s |

The composition of each batch is provided in Table 5. The general procedure used for preparation of the dosage form is provided below:

Procedure: As mentioned in the examples (i.e. Example Nos. 14-19), suitable quantity of Cefepime/Cefepime Hydrochloride/Cefepime Hydrochloride Hydrate, sulbactam sodium and L-arginine were mixed under aseptic and clean conditions and proceeded according to the preparation process sequence of dry powder injection preparation to prepare 10 units of powder injection to be reconstituted with suitable solvents like sterile water for injection, normal saline, 5% dextrose before administration.

### Example 20-21

TABLE 6
Cefdinir and Sulbactam sodium suspension.

| S. | | Example 20 | Example 21 |
|---|---|---|---|
| No | Ingredients | Each 5 ml contains | Each 5 ml contains |
| 1 | Cefdinir | 130 | 130 |
| 2 | Sulbactam sodium | 136.79 | 273.58 |
| 3 | Sucrose | 2308.6 | 2172 |
| 4 | Citric acid | 0.3 | 0.5 |
| 5 | Sodium citrate | 0.1 | 0.2 |
| 6 | Sodium benzoate | 0.2 | 0.2 |
| 7 | Xanthan gum | 5 | 5 |
| 8 | Guar gum | 1 | 1 |
| 9 | Colloidal silicon dioxide | 8 | 8 |
| 10 | Dry flavour | 8 | 8 |
| 11 | Magnesium stearate | 2 | 2 |
| | | 2599.99 | 2600.48 |

The composition of each batch is provided in Table 6. The general procedure used for preparation of the dosage form is provided below:

Procedure: Cefdinir, sulbactam sodium, sucrose, citric acid, sodium citrate, sodium benzoate, xanthan gum, guar gum, Colloidal silicon dioxide were sifted through ASTM mesh # 40 and mixed thoroughly using suitable blender. The suitable flavor was incorporated to prepare 5 bottles of powder for oral suspension, which is ready to be reconstituted with water before administration.

### Example 22-23

TABLE 7
Cefditoren and Tazobactam Tablet

| | | Example 22 | Example 23 |
|---|---|---|---|
| Sr. no | Ingredients | Mg/tablet | Mg/tablet |
| 1 | Cefditoren pivoxil | 245.06 | 245.06 |
| 2 | Tazobactam sodium | 218.86 | 437.72 |
| 3 | Cros carmellose sod. | 50 | 60 |
| 4 | Sod. Caseinate | 70 | 75 |
| 5 | D mannitol | 180 | 225 |
| 6 | Sod. tripoly phosphate | 20 | 20 |
| 7 | HPMC | 10 | 10 |
| 8 | HPC | 75 | 100 |
| 9 | Magnesium stearate | 2 | 4 |
| 10 | Cros carmellose sod. | 25 | 35 |
| 11 | Magnesium stearate | 4 | 6 |
| | | 899.92 | 1217.78 |

The composition of each batch is provided in Table 7. The general procedure used for preparation of the dosage form is provided below:

Procedure: Cefditoren pivoxil, tazobactam sodium, cros carmellose sodium, Sodium caseinate, D mannitol, Sodium tripoly phosphate, HPMC, HPC were sifted through ASTM mesh # 40 and mixed thoroughly using suitable blender. The blend was compacted to get the granules. The granules were further lubricated with magnesium stearate and cross carmellose sodium. The granules so obtained can be compressed into tablets or filled in pouch.

### Example 24-27

TABLE 8
Cefixime and Sulbactam tablets

| | | Example 24 | Example 25 | Example 26 | Example 27 |
|---|---|---|---|---|---|
| Sr. no. | Ingredients | Mg/tablet | Mg/tablet | Mg/tablet | Mg/tablet |
| 1 | Cefixime trihydrate | 223.81 | 223.81 | 447.62 | 447.62 |
| 2 | Sulbactum Sodium | 218.86 | 437.72 | 437.72 | 875.44 |
| 3 | MCC | 100 | 150 | 300 | 400 |
| 4 | Cros carmellose sodium | 40 | 60 | 160 | 220 |
| 5 | HPC | 35 | 45 | 90 | 150 |
| 6 | Colloidal silicon dioxide | 3 | 5 | 10 | 20 |
| 7 | Magnesium stearate | 3 | 4 | 6 | 12 |
| 8 | Colloidal silicon dioxide | 5 | 7 | 15 | 20 |
| 9 | Magnesium stearate | 7 | 9 | 14 | 20 |
| | | 635.67 | 941.53 | 1480.34 | 2165.06 |

The composition of each batch is provided in Table 8. The general procedure used for preparation of the dosage form is provided below:

Procedure: Cefixime trihydrate, Sulbactum Sodium, MCC, cros carmellose sodium, HPC, Colloidal silicon dioxide and magnesium stearate were sifted through ASTM mesh # 40 and mixed thoroughly using suitable blender. The blend was compacted to get the granules. The granules were further lubricated with magnesium stearate and cross carmellose sodium. The granules so obtained can be compressed into tablets or filled in pouch.

### Example 28-31

TABLE 9
Cefpodoxime and Sulbactam tablets

| | | Example | | Example | |
|---|---|---|---|---|---|
| Sr. | | 28 | Example 29 | 30 | Example 31 |
| No | Ingredients | Mg/tablet | Mg/tablet | Mg/tablet | Mg/tablet |
| 1 | Cefpodoxime | 134.5 | 134.5 | 269 | 269 |
| | proxetil | | | | |
| 2 | Sulbactam sod | 109.43 | 218.86 | 218.86 | 437.72 |
| 3 | Sod. Lauryl | 10 | 10 | 20 | 20 |
| | sulfate | | | | |
| 4 | Lactose | 100 | 125 | 200 | 300 |
| | mono hyd | | | | |
| 5 | Calcium CMC | 50 | 75 | 75 | 100 |
| 6 | HPC(LH-11) | 40 | 55 | 55 | 60 |
| 7 | HPMC | 10 | 10 | 10 | 10 |
| 8 | Mag stearate | 1 | 2 | 3 | 6 |
| 9 | HPC(LH-11) | 20 | 30 | 30 | 30 |
| 10 | Mag stearate | 3 | 4 | 6 | 10 |
| | | 477.93 | 664.36 | 886.86 | 1242.72 |

The composition of each batch is provided in Table 9. The general procedure used for preparation of the dosage form is provided below:

Cefpodoxime proxetil, sulbactam sodium, sodium lauryl sulfate, lactose mono hydrate, Calcium CMC, HPC, HPMC, magnesium stearate were sifted through ASTM mesh # 40 and mixed thoroughly using suitable blender. The blend was compacted to get the granules. The granules were further lubricated with magnesium stearate and cross carmellose sodium. The granules so obtained can be compressed into tablets or filled in pouch.

### Example 32-35

TABLE 10
Cefprozil and Sulbactam tablet

| Sr. | | Example 32 | Example 33 | Example 34 | Example 35 |
|---|---|---|---|---|---|
| No | Ingredients | Mg/tablet | Mg/tablet | Mg/tablet | Mg/tablet |
| 1 | Cefprozil monohydrate | 264.41 | 264.41 | 528.82 | 528.82 |
| 2 | sulbactam sodium | 273.58 | 547.12 | 547.12 | 1094.2 |
| 3 | MCC | 100 | 150 | 200 | 300 |
| 4 | Methocel | 10 | 10 | 20 | 20 |
| 5 | sod. starch glucolate | 35 | 60 | 70 | 120 |
| 6 | HPC | 40 | 50 | 80 | 100 |
| 7 | Magnesium stearate | 3.5 | 5 | 7 | 10 |
| 8 | sodium. starch glucolate | 10 | 20 | 20 | 40 |
| 9 | Magnesium stearate | 4 | 6 | 8 | 12 |
| | total | 740.49 | 1112.53 | 1480.94 | 2225.02 |

The composition of each batch is provided in Table 10. The general procedure used for preparation of the dosage form is provided below:

Procedure: Cefprozil monohydrate, sulbactam sodium, MCC, Methocel, sodium Starch glucolate, HPC, magnesium stearate, sodium starch glycolate were sifted through ASTM mesh # 40 and mixed thoroughly using suitable blender. The blend was compacted to get the granules. The granules were further lubricated with magnesium stearate and cross carmellose sodium. The granules so obtained can be compressed into tablets or filled in pouch.

### Example 36

TABLE 11
Ceftibuten and Sulbactum Capsule

| Sr. | | Example 36 |
|---|---|---|
| No | Ingredients | Mg/capsule |
| 1 | Ceftibuten Dihydrate | 435.11 |
| 2 | Sulbactum Sodium | 437.72 |
| 3 | Microcrystalline Cellulose | 252 |
| 4 | Sodium Starch Glycolate | 40 |
| 5 | Magnesium Sterate | 5 |
| 6 | Sodium Starch Glycolate | 10 |
| 7 | Magnesium Sterate | 6 |
| | | 750.72 |

The composition of each batch is provided in Table 11. The general procedure used for preparation of the dosage form is provided below:

Procedure: Ceftibuten Dihydrate, sulbactum sodium, microcrystalline cellulose, sodium starch glycolate, magnesium sterate, sodium starch glycolate, magnesium sterate were sifted through ASTM mesh # 40 and mixed thoroughly using suitable blender. The blend was compacted to get the granules. The granules were further lubricated with magnesium stearate and cross carmellose sodium. The granules so obtained can be filled in pouch or capsules of suitable size.

While the present invention has been described in terms of its specific embodiments, certain modifications and equivalents will be apparent to those skilled in the art and are intended to be included within the scope of the present invention.

We claim:

1. A method of inhibiting bacterial infections caused by resistant Extended-spectrum β-lactamase producing strains (ESBLs) in a warm blooded animal, the method comprising administering a combination of 2 gm of cefepime and 0.1-4 gm of sulbactam to the warm blooded animal.

2. A pharmaceutical composition comprising 2 gm of cefepime in combination with 0.1-4 g of sulbactam, and one or more pharmaceutically acceptable excipients.

3. The pharmaceutical composition according to claim 2, wherein the cefepime is present in the form of cefepime internal salt, cefepime hydrochloride or cefepime hydrochloride hydrate, or addition agents.

4. The pharmaceutical composition according to claim 3, wherein the addition agent is L-arginine.

5. The pharmaceutical composition according to claim 2, wherein the sulbactam is present in the form of sulbactam sodium or potassium salt.

6. The pharmaceutical composition according to claim 2, wherein the composition is for parenteral administration.

7. The pharmaceutical composition according to claim 2, wherein the pharmaceutically acceptable excipient comprises one or more of antioxidants, buffers, preservatives, tonicity adjusting agents, and chelating agents.

8. The pharmaceutical composition according to claim 7, wherein the antioxidant comprises one or more of butylated hydroxytoluene (BHT), ascorbic acid, sodium bisulphite, sodium metabisulphite, and mixtures thereof.

9. The pharmaceutical composition according to claim 7, wherein the buffer comprises one or more of citrates, acetates, borax, phosphates, and mixtures thereof.

10. The pharmaceutical composition according to claim 7, wherein the preservative comprises one or more of benzyl alcohol, methyl paraben, propyl paraben, benzyl paraben, and mixtures thereof.

11. The pharmaceutical composition according to claim 7, wherein the tonicity adjusting agent comprises one or more of dextrose, sodium chloride, mannitol, and mixtures thereof.

12. The pharmaceutical composition according to claim 7, wherein the chelating agent comprises one or more of sodium ethylene-diamine-tetra-acetic acid (EDTA), citric acid and mixtures thereof.

13. A method of treating a resistant bacterial infection caused by Extended-spectrum β-lactamase producing strains (ESBLs) in a warm blooded animal, the method comprising providing a dosage form to the warm blooded animal comprising cefepime in combination with sulbactam via parenteral route, followed by providing a dosage form that includes an oral third generation cephalosporin with a suitable β lactamase inhibitor.

14. The method according to claim 13, wherein the oral third generation cephalosporin and β lactamase inhibitor are present in a weight ratio of 1:1 to 1:4.

15. The method according to claim 13, wherein the oral third generation cephalosporin comprises one or more of cefdinir, cefditoren, cefixime, cefpodoxime, cefprozil, and ceftibuten.

16. The method according to claim 13, wherein the suitable β lactamase inhibitor comprises one or both of sulbactam and tazobactam.

17. The method according to claim 13, wherein the dosage form of oral third generation cephalosporin comprises a tablet, powder, capsule or granules to be reconstituted before administration.
